(19) United States
(12) Patent Application Publication
(10) Pub. No.: US 2017/0157045 A1
(43) Pub. Date: Jun. 8, 2017

(54) APPARATUS AND METHOD FOR GENERATING HYDROGEN-CONTAINING LIQUID

(71) Applicant: MiZ Company Limited, Kanagawa (JP)

(72) Inventors: Ryousuke KUROKAWA, Kanagawa (JP); Fumitake SATOH, Kanagawa (JP); Bunpei SATOH, Kanagawa (JP)

(21) Appl. No.: 15/367,696

(22) Filed: Dec. 2, 2016

(30) Foreign Application Priority Data
Dec. 2, 2015 (JP) 2015-235621
May 11, 2016 (JP) 2016-095438

(51) Int. Cl. A61K 9/00; A61K 9/48; A61K 33/00

(52) U.S. Cl. CPC A61K 9/0095; A61K 33/00; A61K 9/4808

(57) ABSTRACT

An object is to provide an apparatus and method for generating a hydrogen-containing liquid that are capable of generating a hydrogen-containing liquid of which the hydrogen concentration after 10 minutes exceeds 6 ppm and the hydrogen concentration after 24 hours exceeds 10 ppm. The apparatus comprises a hydrogen generating agent (11) reacting with water to generate hydrogen gas, a capsule (20) configured to be charged with the hydrogen generating agent (11) and discharge the hydrogen gas generated in the capsule (20) to external, and a container (3) configured to be charged with a liquid (L) that is an object to which the hydrogen gas generated in the capsule (20) is added. The ratio (V/W) of a volume (V ml) of the capsule (20) to a weight (W g) of the hydrogen generating agent (11) is 11.4 or less and more preferably 8.2 or less.

### BACKGROUND OF THE INVENTION

1. Technical Field of the Invention

The present invention relates to an apparatus and method for generating a liquid that contains hydrogen.

2. Description of the Related Art

The present applicant has previously proposed hydrogen adding equipment for living organism applicable fluid (Patent Document 1: JP4652479B). This equipment includes a hydrogen generating system, such as using aluminum, which reacts with water to generate hydrogen gas and a hydrogen bubble forming implement which has a one-way valve and houses the hydrogen generating system.

Patent Document 1: JP4652479B

A number of papers and other literature have reported that effects such as inhibition of oxidant stress can be obtained by taking a hydrogen-containing liquid into a living body. However, a quantitative limit exists, such as when the liquid is taken into the body by drinking. Therefore, development of an apparatus is demanded which can generate a liquid that contains high-concentration hydrogen even in the same amount of the liquid. In the above previously-proposed prior art, the present applicant has successfully generated a hydrogen-containing liquid of which the hydrogen concentration after 10 minutes is 5 ppm and the hydrogen concentration after 24 hours is 7 ppm, but has not yet generated a hydrogen-containing liquid of which the hydrogen concentration after 10 minutes exceeds 6 ppm and the hydrogen concentration after 24 hours exceeds 10 ppm under the same condition.

An object of the present invention is, therefore, to provide an apparatus and method for generating a hydrogen-containing liquid that are capable of generating a hydrogen-containing liquid of which the hydrogen concentration after 10 minutes exceeds 6 ppm and the hydrogen concentration after 24 hours exceeds 10 ppm.

### SUMMARY OF THE INVENTION

The present invention achieves the above object by providing an apparatus for generating a hydrogen-containing liquid. The apparatus comprises a hydrogen generating agent that reacts with water to generate hydrogen gas, a capsule configured to house the hydrogen generating agent and discharge the hydrogen gas generated in the capsule to external, and a container configured to store a liquid that is an object to which the hydrogen gas generated in the capsule is added. The ratio (V/W) of a volume (V ml) of the capsule to a weight (W g) of the hydrogen generating agent is 11.4 or less in an embodiment and is more preferably 8.2 or less in another embodiment.

According to the present invention, a hydrogen-containing liquid of which the hydrogen concentration after 10 minutes exceeds 6 ppm and the hydrogen concentration after 24 hours exceeds 10 ppm can be generated.

### BRIEF DESCRIPTION OF DRAWINGS

FIG. 1 is a schematic view illustrating constitutional components of an apparatus for generating a hydrogen-containing liquid according to an embodiment of the present invention;

FIG. 2A is a perspective view illustrating a capsule of FIG. 1;

FIG. 2B is a cross-sectional view along line IIB-IIB of FIG. 2A;

FIG. 2C is a cross-sectional view along line IIC-IIC of FIG. 2A;

FIG. 2D is a cross-sectional view along line IID-HD of FIG. 2B;

FIG. 3A is a view illustrating a method of using the apparatus for generating a hydrogen-containing liquid illustrated in FIG. 1;

FIG. 3B is a view illustrating the method of using the apparatus for generating a hydrogen-containing liquid illustrated in FIG. 1;

FIG. 3C is a view illustrating the method of using the apparatus for generating a hydrogen-containing liquid illustrated in FIG. 1;

FIG. 3D is a view illustrating the method of using the apparatus for generating a hydrogen-containing liquid illustrated in FIG. 1;

FIG. 3E is a view illustrating the method of using the apparatus for generating a hydrogen-containing liquid illustrated in FIG. 1;

FIG. 3F is a view illustrating the method of using the apparatus for generating a hydrogen-containing liquid illustrated in FIG. 1;

FIG. 3G is a view illustrating the method of using the apparatus for generating a hydrogen-containing liquid illustrated in FIG. 1;

FIG. 3H is a view illustrating the method of using the apparatus for generating a hydrogen-containing liquid illustrated in FIG. 1;

FIG. 4 is a schematic view illustrating constitutional components of an apparatus for generating a hydrogen-containing liquid according to another embodiment of the present invention;

FIG. 5 is a view illustrating a method of using the apparatus for generating a hydrogen-containing liquid illustrated in FIG. 4;

FIG. 6 is a graph illustrating Examples 1 and 2 and Comparative Examples 1 and 2 in which a hydrogen-containing liquid was generated using the apparatus and method for generating a hydrogen-containing liquid according to the present invention; and

FIG. 7 is a graph illustrating Examples 3 and 4 and Comparative Examples 3 and 4 in which a hydrogen-containing liquid was generated using the apparatus and method for generating a hydrogen-containing liquid according to the present invention.

### DESCRIPTION OF THE PREFERRED EMBODIMENT

Hereinafter, an apparatus and method for generating a hydrogen-containing liquid according to an embodiment of the present invention will be described. Apparatus 1 for generating a hydrogen-containing liquid according to the present embodiment comprises a hydrogen generating agent 11 that reacts with water to generate hydrogen gas, a capsule 20 that has a one-way valve 21 and is configured to be charged with the hydrogen generating agent 11, and a container 30 configured to be charged with a liquid L that is an object to which the hydrogen gas generated in the capsule 20 is added. The one-way valve 21 (which may also be a return check valve or gas-permeable film) allows the hydrogen gas generated in the capsule 20 to be discharged to the external but blocks the external liquid from entering the capsule 20.

The liquid L in the present embodiment encompasses all of liquids that can be an object into which hydrogen molecules are dissolved using the apparatus 1 of the present embodiment. Examples of the liquid L include water and aqueous solution as well as various beverages such as drinking water, tea and coffee. Examples of the liquid L further include normal saline solution prepared in terms of osmolality for use as injection, intravenous drip, transfusion and the like; injection solution prepared for resupply of nutrition and electrolytes; injection solution in which a medical agent is dissolved; blood preparation (blood for blood transfusion) and own blood to be used for blood transfusion; enteral solution; and organ preservative liquid prepared to preserve organs. In particular, the liquid L in the present embodiment encompasses a liquid that can be applied to living organisms such as plants and animals including human. Hydrogen is dissolved into the liquid L of such a kind to obtain a hydrogen-containing liquid, which is then applied to living organisms via inhalation or atomization through the mouth or nose, drinking from the mouth, injection to the skin or into the vein or artery, and the like. The active constituent of the hydrogen-containing liquid, in particular of a high-concentration hydrogen-containing liquid including a supersaturated hydrogen-containing liquid, is the hydrogen and the functionality thereof is primarily inhibition of oxidant stress.

The hydrogen generating agent 11 in the present embodiment is a material that reacts with water to generate hydrogen gas. Specifically, the hydrogen generating agent 11 contains a metallic material that has a higher ionization tendency than hydrogen and an accelerant that accelerates the reaction of the metallic material with water. The hydrogen generating agent 11 put in a bag body 12 that is permeable to water is referred to as a hydrogen generating body 10. The metallic material is a substance that reacts with water thereby to generate hydrogen. Examples of the metallic material include an elementary substance of metal that has a higher ionization tendency than hydrogen and also include hydrogenated compounds including a hydrogenated metal. In consideration of good reactivity with water, it is preferred to use metal calcium, calcium hydride, metal magnesium, magnesium hydride, or the like. In consideration of the safety of the resulting reaction products, metal magnesium is particularly preferably used. In consideration of the safety of the resulting reaction products and the food sanitation law, iron, aluminum, nickel and cobalt are preferably used. Among them, metal aluminum is preferably used also in view of the good appearance and cost and the safety in handling.

The bag body 12, which houses the above-described metallic material and accelerant, is composed of a material that is permeable to water. The bag body 12 of the present embodiment is provided to ensure separation between the liquid L and the metallic material and accelerant. Nonwoven fabric and other similar materials may be exemplified as a material of the bag body 12. The bag body 12 is permeable to hydrogen gas and water but nonpermeable to the metallic material, accelerant, and reaction residue thereof. Pore size of the bag body 12 is 1,000 μm or less, preferably 500 μm or less, more preferably 150 μm or less, and particularly preferably 50 μm or less. From the relation with the pore size of the bag body 12, the average particle diameter of the metallic material and accelerant may preferably be a particle diameter with which they do not pass through the bag body 12 to external and the enhanced activity due to microparticulation is expected. For example, the average particle diameter of the metallic material is 3,000 μm or less, preferably 1,000 μm or less, further preferably 500 μm or less, and particularly preferably 250 μm or less.

The hydrogen generating agent 11 of the present embodiment contains the metallic material and, if necessary, may further contain an accelerant, such as a metal-ion sequestering agent and pH adjuster, which accelerates the hydrogen generation reaction.

Examples of the metal-ion sequestering agent which can be used in the present embodiment include substances that are absolutely insoluble or scarcely dissolved in water and generate substances having properties of adsorbing metal ions in the capsule 20 or in the bag body 12. Insoluble or hardly-soluble metal-ion sequestering agents may preferably be used, such as a cation-exchange resin. Among them, a hydrogen ion-type cation-exchange resin is further preferred because it also has functionality as a pH adjuster. The hydrogen ion-type cation-exchange resin includes an acidic cation-exchange resin having a sulfonic acid group as the exchange group or an acidic cation-exchange resin having a carboxylic acid group as the exchange group, which adsorbs metal ions and releases hydrogen ions (H⁺).

Examples of the pH adjuster which can be used in the present embodiment include a substance that has a property of inhibiting (neutralizing or preventing the generation of) hydroxide ions (OH⁻) by supplying hydrogen ions (H⁺), such as citric acid, adipic acid, malic acid, acetic acid, succinic acid, gluconic acid, lactic acid, phosphoric acid, hydrochloric acid, sulfuric acid and other acids, and also include a substance that is hydrolyzed to form insoluble hydroxide thereby to remove hydroxide ions. A pH adjuster that is hydrolyzed to form insoluble hydroxide, such as a mineral ore containing aluminum ions, may preferably be used. Among them, alums such as aluminum ammonium sulfate may be further preferred because they are hydrolyzed to generate insoluble aluminum hydroxide and also have functionality as a metal-ion sequestering agent (aggregating agent) for magnesium ions and calcium ions. As described above, the hydrogen ion-type cation-exchange resin and alums are more preferred substances because they have both the functionality as a metal-ion sequestering agent and the functionality as a pH adjuster even by one agent.

An acid or alkaline agent can be used as the accelerant which accelerates the hydrogen generation reaction of the metallic material. Examples of the acid include, but are not limited to, an acid that generates a solid precipitate after the reaction and a solid acid such as ion-exchange resin. When an amphoteric metal such as aluminum and zinc is used as the hydrogen generating agent, an alkaline agent such as calcium hydroxide, calcium oxide and anion-exchange resin can also be used other than the acid. Among them, an alkaline agent as a food additive may preferably be used, such as calcium hydroxide (lime hydrate), calcined lime (calcium oxide), calcined calcium, magnesium oxide, magnesium hydroxide, and anion-exchange resin. The hydrogen generation reaction accelerant, which reacts with a metal such as aluminum as a food additive having a higher ionization tendency than hydrogen to generate a precipitate, can suppress re-dissolution of ions of the metal after the hydrogen generation reaction and thus does not substantially change properties of the liquid L to be applied to a living organism.

To suppress the time degradation of the metallic material, the hydration number and water content ratio of the substances such as the metal-ion sequestering agent and pH adjuster contained in the hydrogen generating system may preferably be low. More specifically, with respect to the hydration number, they may be trihydrate or lower, preferably dihydrate or lower, more preferably monohydrate or lower, and particularly preferably nonhydrate or anhydride. The water content ratio may be 40 wt % or less, preferably 30 wt % or less, more preferably 20 wt % or less, and particularly preferably 15 wt % or less.

The metallic material of the present embodiment is brought into contact with water to generate hydrogen gas in the capsule 20. Examples of such water include, but are not limited to, tap water, filtered water, ion-exchanged water, purified water, pure water, and RO water. The above-described liquid L itself can also be used as the water. The term “water” as used herein also encompasses gas and vapor such as water vapor. Regardless of the components, hardness and liquid properties, any liquid or gas that contains water can be used as the water of the present embodiment.

A rough target of the amount of water to be reacted with the hydrogen generating agent 11 which contains a metallic material may preferably be a small amount to such an extent that the water does not remain in the capsule 20 which houses the hydrogen generating body 10, such as when the bag body 12 as a whole can be immersed into the liquid L in a moment, as will be described later. For example, the amount of water remaining in the capsule 20 may be 10 cc or less, preferably 5 cc or less, further preferably 3 cc or less, and particularly preferably 1 cc or less. To prevent such excessive water from flowing into the capsule 20 from the bag body 12, the capsule 20 or the bag body 12 may preferably incorporate therein a water-absorbing substance or material, such as water-absorbing beads, ion-exchange resin (dry ion-exchange resin is further preferred because it has high water-absorbing ability), water-absorbing paper, hyaluronan, and polyacrylic.

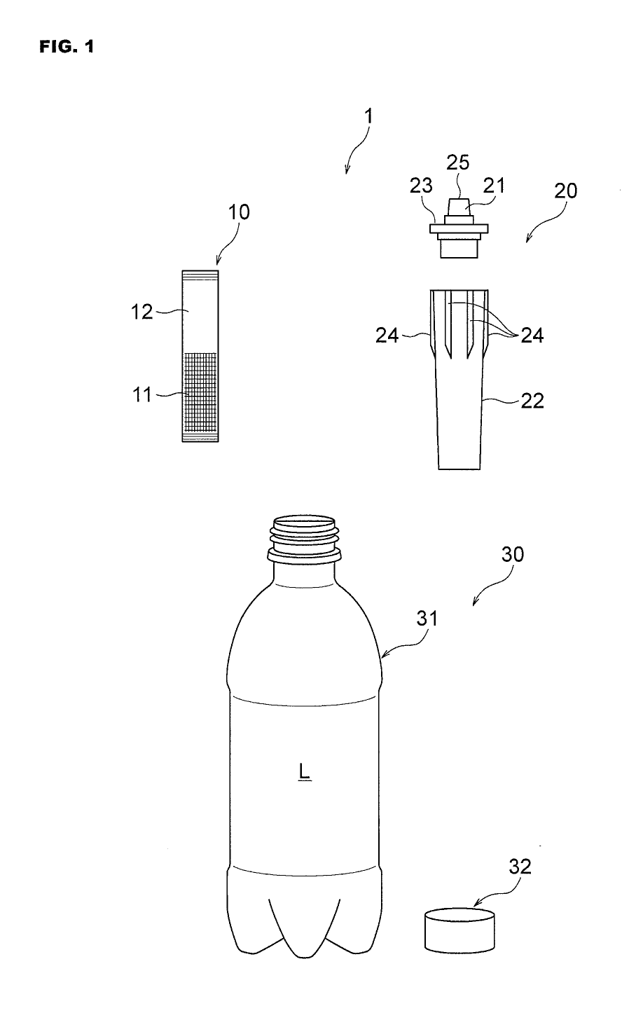

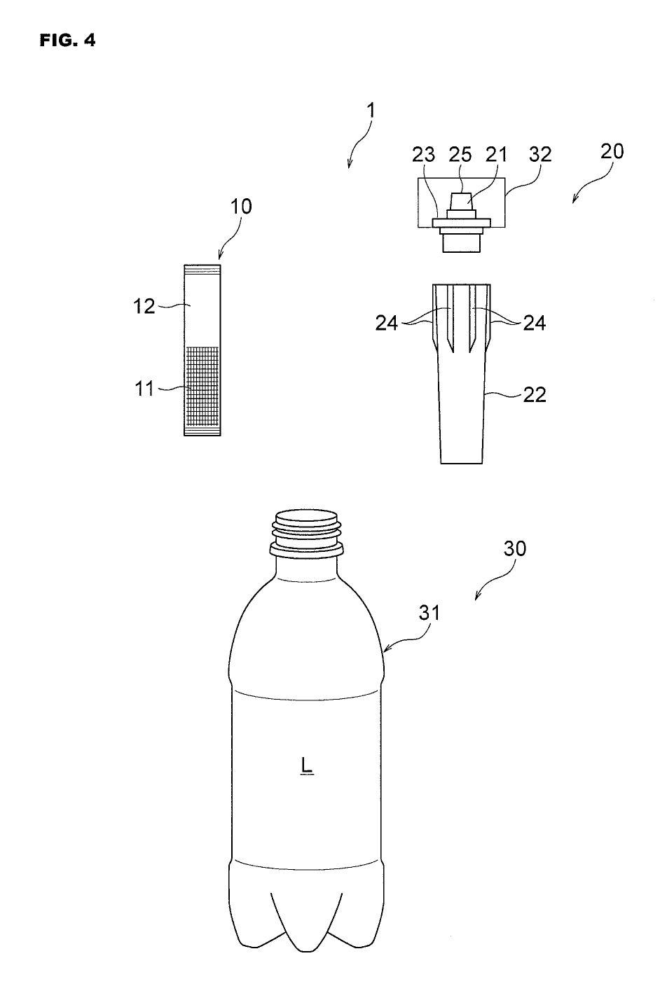

The capsule 20 of the present embodiment is configured to separate the liquid L and the hydrogen generating agent 11 from each other and feed the hydrogen gas generated in the hydrogen generating body 10 into the container 30, which stores the liquid L, via the one-way valve 21 of the capsule 20. The apparatus 1 of the present embodiment including the capsule 20 may be provided as a separate member from the container 30 and housed in the container 30 which stores the liquid L, or may also be provided as a structural part that is preliminarily incorporated in the container 30. FIG. 1 illustrates an embodiment in which the capsule 20 is configured as a separate member from the container 30 and FIG. 4 illustrates an embodiment in which the capsule 20 is incorporated as a part of the container 30. Details thereof will be described below.

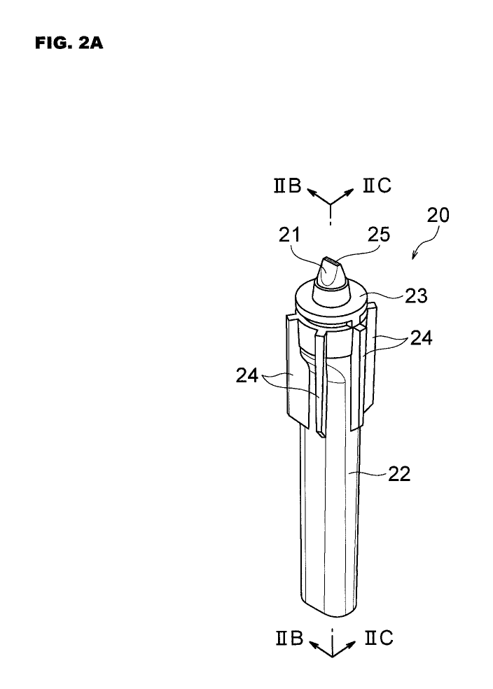

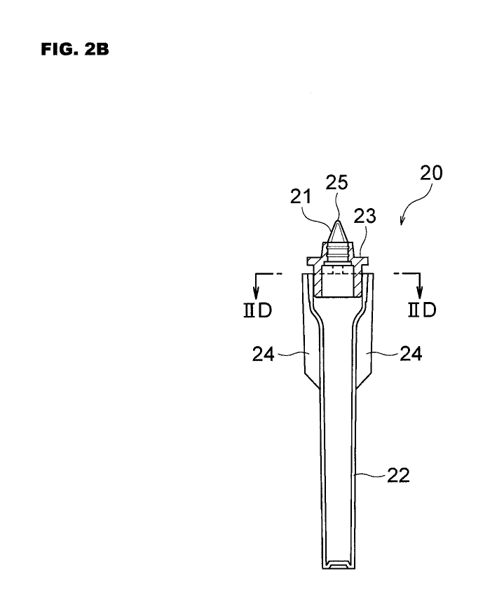

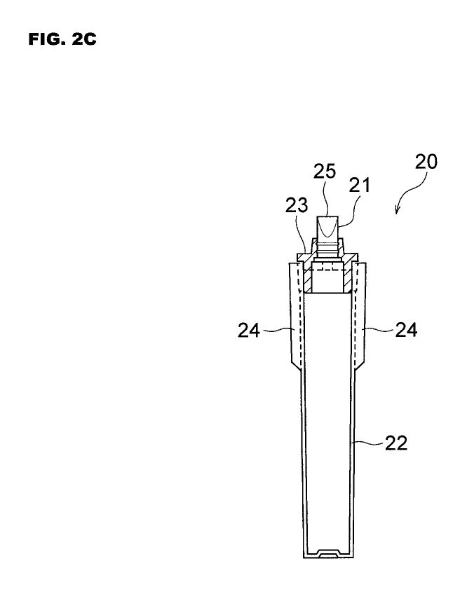

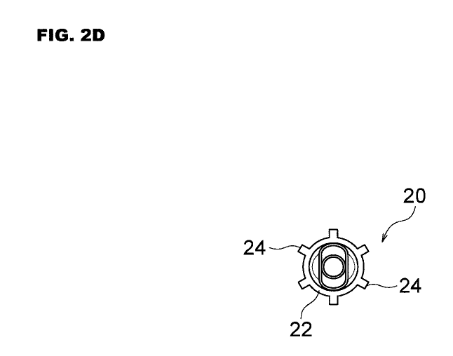

FIGS. 2A to 2D illustrate a first embodiment of the capsule 20. The capsule 20 of the present embodiment comprises a capsule main body 22, a valve cap 23, and a duckbill valve 21. The capsule main body 22 is a bottom-closed tubular member formed of a heat-resistant resin material that can withstand the reaction heat of the hydrogen generating agent 11 and water. The hydrogen generating body 10 is put into the capsule main body 22 and the valve cap 23 is fitted with an upper opening of the capsule main body 22. Outer surface of the capsule main body 22 is formed integrally with a plurality of projections 24 that are provided at certain intervals along the circumferential direction. These projections 24 provide a structure for preventing the deformation and the like of the container 30 due to the reaction heat of the hydrogen generating agent 11 and water which transfers from the capsule main body 22 to the container 30 (the reaction heat may possibly be a high temperature of about 200° C. depending on the type of the metallic material). In other words, the projections 24 are provided to elongate the contact distance and/or reduce the contact area between the capsule 20 and the container 30 thereby to suppress the heat transferring to the container 30. The projections 24 are provided at the outer surface of the capsule main body 22 in the embodiment illustrated in FIGS. 2A to 2D, but may also be provided at the outer surface of the valve cap 23.

The valve cap 23 of the capsule 20 of the present embodiment has functions to hold the duckbill valve 21 and close the upper opening of the capsule main body 22. Like the capsule main body 22, the valve cap 23 is formed of a heat-resistant resin material that can withstand the reaction heat of the hydrogen generating agent 11 and water.

The duckbill valve 21 is molded from a resin material having elasticity into a bird's bill-like shape and a slit 5 is provided in the ridge line portion at the tip of the duckbill valve 21. According to this structure, the slit 25 is in a state of being closed due to the own elasticity of the duckbill valve 21 until the inner pressure of the capsule 20 becomes sufficiently high, and the sufficiently increased inner pressure then expands the slit of the duckbill valve 21 against the own elasticity to discharge the hydrogen gas. The duckbill valve 21 is one example of the one-way valve or return check valve of the present invention.

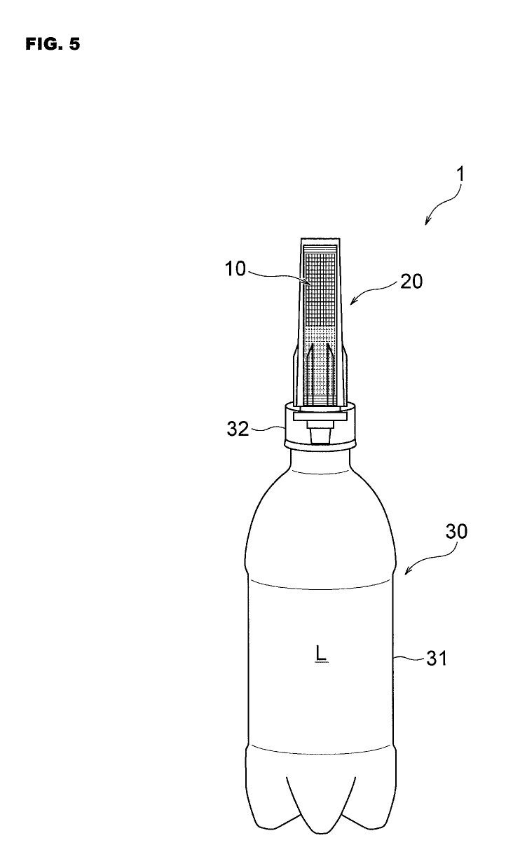

In an alternative embodiment, a gas-permeable film may be provided as substitute for the duckbill valve 21 which is one example of the one-way valve or return check valve. The gas-permeable film allows the hydrogen gas generated in the capsule 20 to be discharged to external of the capsule 20, but blocks the external liquid from entering the capsule 20. As will be described in another embodiment of FIGS. 4 and 5, the capsule 20 may be fixed to a cap 32 of the container 30 so as to be provided outside the container 30, but in an alternative embodiment the capsule 20 may be fixed to the cap 32 so as to be provided inside the container 30. When the mixing of the hydrogen generating agent 11 into the liquid L in the container 30 is not problematic, the valve structure is not limited to the one-way valve, return check valve, gas-permeable film, etc. and may be one that allows the hydrogen gas generated in the capsule 20 to be discharged to external of the capsule 20 and also allows the external liquid to enter the capsule 20.

The container 30 of the present embodiment is a container for storing the above-described liquid L and examples thereof include a tightly-closed container that is configured not to expose the contents to the air. Examples of the tightly-closed container include those with lids, such as PET bottles and aluminum bottles with caps. It is preferred that the tightly-closed container 30 have a portable form and volume in order for a person to easily shake it in the hand. The volume of the tightly-closed container 30 may be, but is not limited to, 2 L or less, preferably 1 L or less, and particularly preferably 0.5 L or less.

Preferred materials for the tightly-closed container 30 are to have low hydrogen permeability. This is because, the lower the hydrogen permeability is, the less likely the generated hydrogen is to escape outside the container 30.

The hydrogen permeability of the container 30 is measured as follows. That is, with reference to a method described in JP2009-221567A and the like, hydrogen-dissolved water is generated to stably maintain approximately the saturated concentration (1.6 ppm at 20° C. and 1 atm) with a volume of 20 times the inner volume of a tightly-closed container as an object to be measured, and the tightly-closed container is then immersed in the hydrogen-dissolved water for five hours after being fully filled with filtrated water (such as charcoal-treated water obtained by treating Fujisawa city tap water to pass through a charcoal column). Thereafter, the dissolved hydrogen concentration in the filtrated water is measured. When the dissolved hydrogen concentration is 1,000 ppb or lower, preferably 500 ppb or lower, more preferably 100 ppb or lower, and particularly preferably 10 ppb or lower, the container 30 is included in examples of the container having low hydrogen permeability according to the present embodiment.

The container 30 of the present embodiment may preferably have pressure-resistant performance capable of withstanding an increased inner pressure due to the generation of hydrogen, in addition to the tightly-closing ability. Specifically, the container 30 may be a pressure-resistant container that can withstand an inner pressure of 0.11 MPa, preferably 0.4 MPa, further preferably 0.5 MPa, and particularly preferably 0.8 MPa, as the absolute pressure. A PET bottle for carbonated drink and the like may preferably be used. The mouth part of container 30 of the present embodiment may preferably be provided with a mechanism (vent slot) for releasing the pressure in the course of opening the cap so that safety opening can be performed.

The hydrogen-containing liquid obtained in the present embodiment is a hydrogen-containing liquid of which the dissolved hydrogen concentration is 8 ppm or higher and preferably 10 ppm or higher. In the present embodiment, the supersaturated hydrogen-containing liquid refers to a high-concentration hydrogen-containing liquid of which the dissolved hydrogen concentration is not lower than the solubility at ordinary temperatures and pressures (1.6 ppm), in particular the dissolved hydrogen concentration is 8.0 ppm or higher in an embodiment, 9.0 ppm or higher in another embodiment, and 10.0 ppm or higher in still another embodiment.

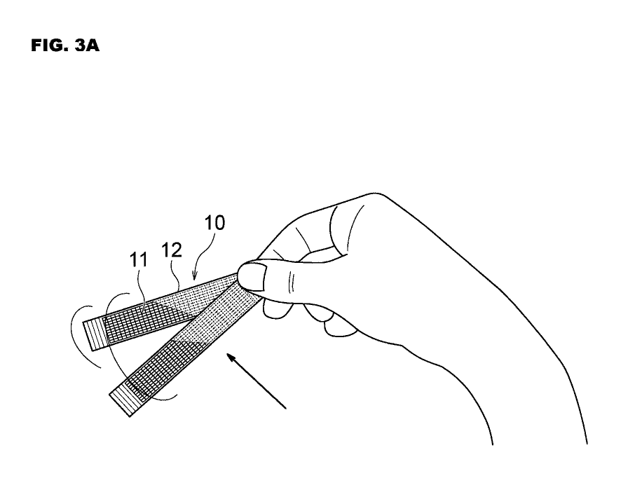

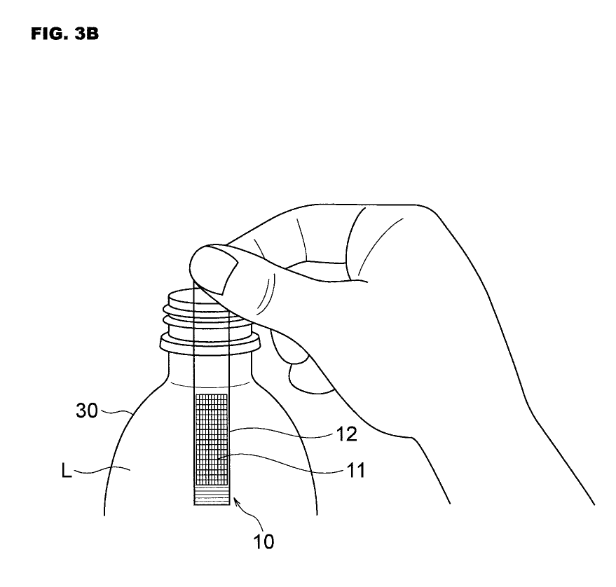

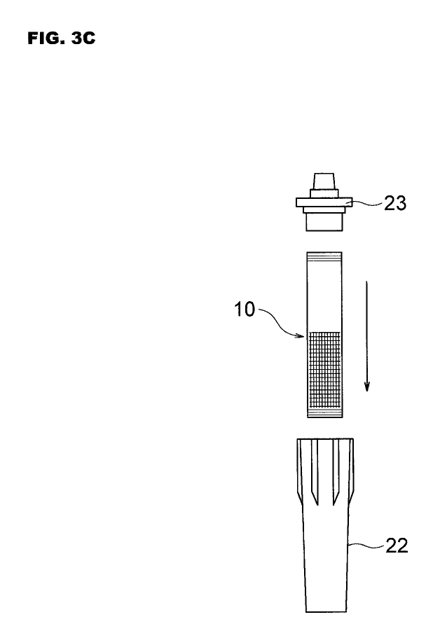

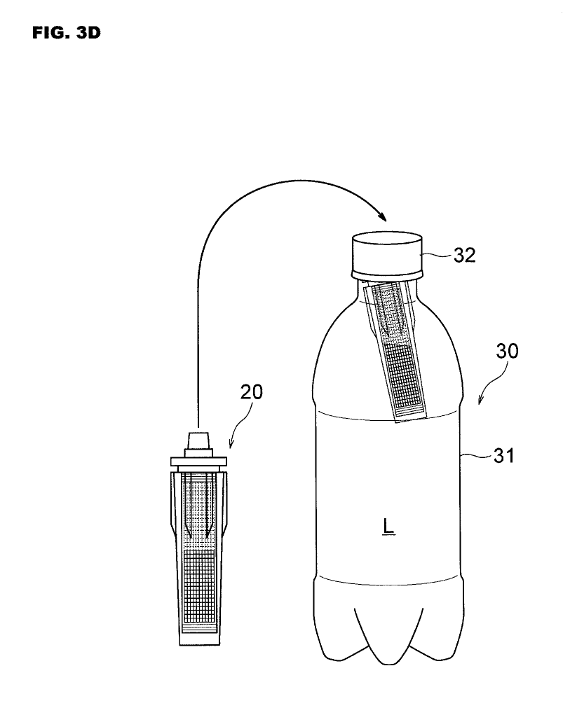

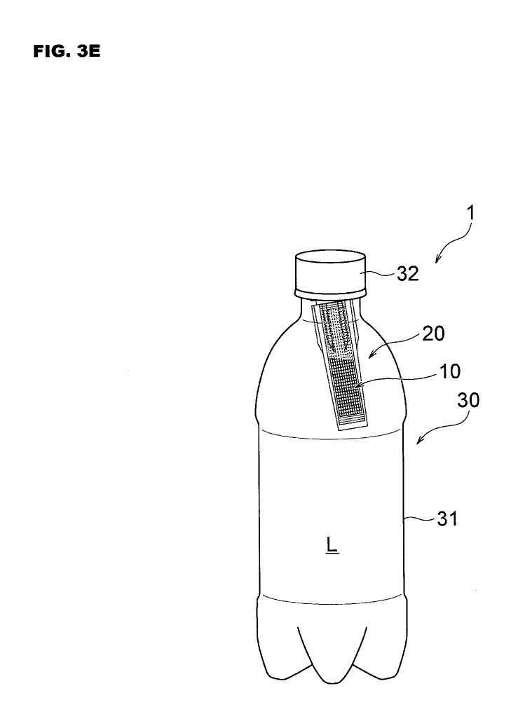

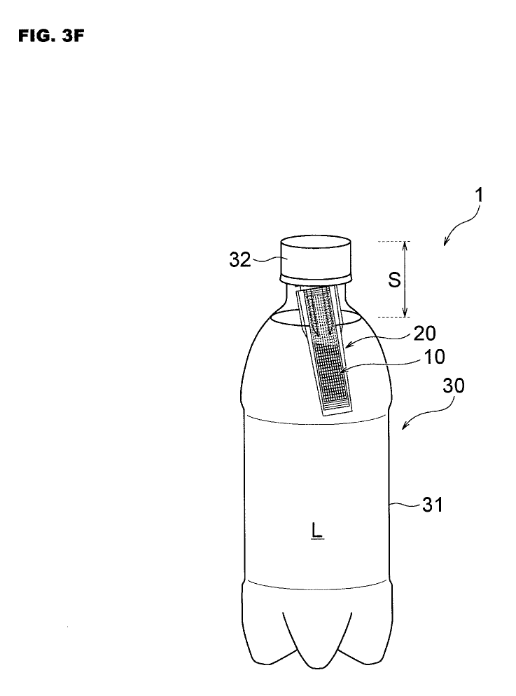

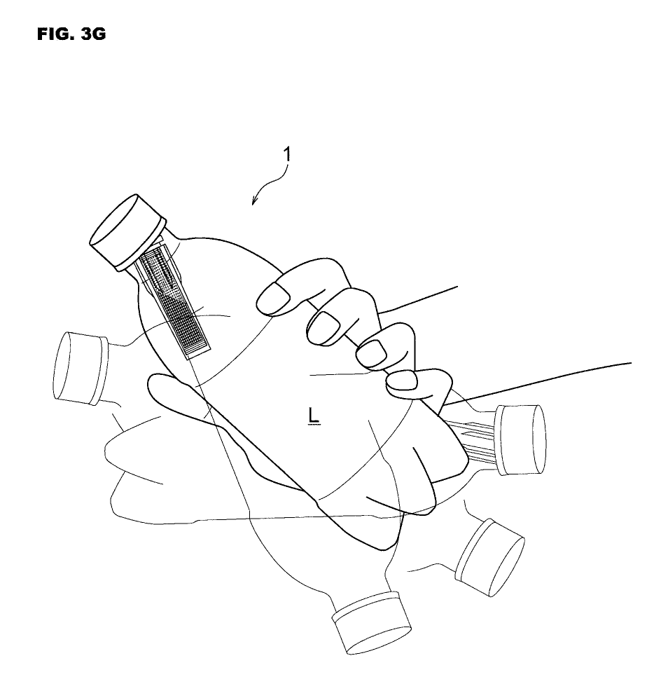

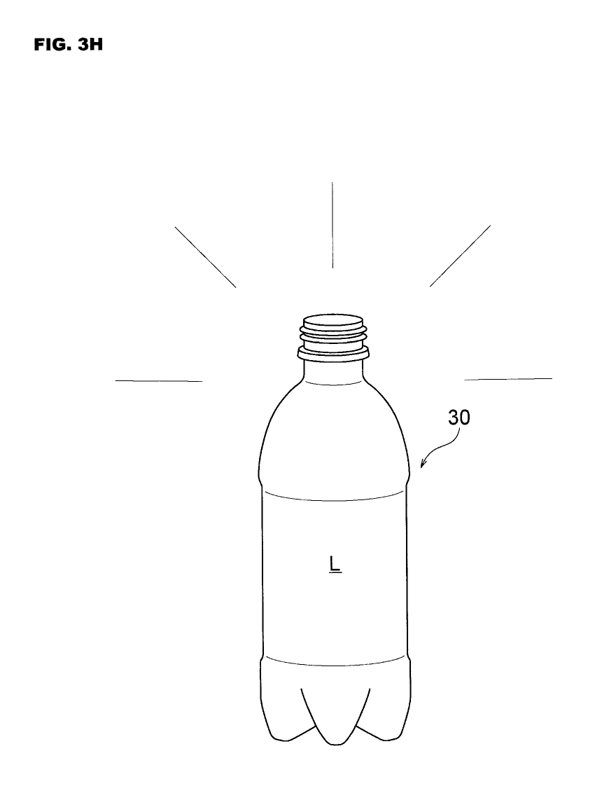

A method of using the apparatus 1 for generating a hydrogen-containing liquid according to the present embodiment will now be described with reference to FIGS. 3A to 3H. First, as illustrated in FIG. 3A, the hydrogen generating body 10 is prepared. The hydrogen generating body 10 is configured such that the bag body 12 is charged with the hydrogen generating agent 11 which contains a metallic material, such as aluminum and magnesium, and an accelerant. Then, as illustrated in FIG. 3B, the hydrogen generating body 10 is immersed in a liquid L stored, for example, in the container 30 for five to six seconds so that the bag body 12 gets wet. Next, as illustrated in FIG. 3C, the hydrogen generating body 10 thus wet ted is put into the capsule main body 22, which is then closed with the valve cap 23. The container 30 is filled with the liquid L fully to the upper opening. That is, the container 30 is filled with the liquid L to reduce the volume of air in the head space S of the container 30 as much as possible.

Subsequently, as illustrated in FIG. 3D, the capsule 20 is put into the container 30, which is then closed with the cap 32. The capsule 20 of the present embodiment floats on the liquid surface in the container 30 because the specific gravity of the capsule 20 is smaller than that of the liquid L, but in an alternative embodiment, a capsule of a larger specific gravity than that of the liquid L may be used to sink in the liquid L in the container 30. After a while, the hydrogen generating agent 11 starts to react with water thereby to generate hydrogen gas in the capsule 20. When the inner pressure of the capsule 20 increases to some extent and overcomes the own elasticity of the duckbill valve 21, the increased inner pressure opens the slit 25 of duckbill valve 21, from which the hydrogen gas is discharged. However, the liquid L does not enter the capsule 20 from the opened slit 25 because of the increased inner pressure.

The hydrogen gas is discharged from inside of the capsule 20 via the duckbill valve 21 into the container 30 and then, as illustrated in FIGS. 3E and 3F, accumulates in the head space S of the container 30 (space in the vicinity of the upper opening of the container 30). During this operation, the capsule 20 is at a relatively high temperature due to the reaction heat of the hydrogen generating agent 11 and water, but the heat of high temperature is difficult to transfer to the container 30 because the projections 24 come into contact with the container 30 thereby to avoid direct contact between the capsule main body 22 and the container 30. This can prevent deformation and the like of the container 30.

The hydrogen gas generated by the reaction of the hydrogen generating agent 11 and water in the capsule 20 is released via the duckbill valve 21 into the container 30 storing the liquid L and forms a hydrogen gas phase of high pressure and high concentration in the head space S. Even when the apparatus 1 of the present embodiment is disposed to sink in the liquid L, most of the generated hydrogen molecules first transfer into the air space of the head space in the container 30 without dissolving into the liquid L. When the hydrogen generating body 10, in which the bag body 12 is charged with the hydrogen generating agent 11, is put into the capsule 20, the hydrogen gas is discharged as hydrogen gas bubbles from the duckbill valve 21 only after concentrating in an appropriate amount inside the capsule 20. In other words, when discharged into the liquid L, the hydrogen molecules are released as hydrogen gas bubbles that already have certain dimensions, and the capsule 20 may act as a kind of stopper for the hydrogen gas. It can therefore be inferred that the hydrogen molecules first transfer into the air space of the head space in the container 30 without dissolving into the liquid L.

This can also be visually observed. For example, when the apparatus 1 of the present embodiment is disposed in the container 30 storing the liquid L and the container 30 is left for a while in a laid form, the hydrogen gas generated in the capsule 20 is intermittently released as hydrogen bubbles from the duckbill valve 21 while progressively increasing the volume of the hydrogen gas phase. In other words, the released hydrogen gas moves upward in the water and transfers rapidly into the gas phase of the head space in the tightly-closed container 30 because the released hydrogen gas has a large bubble size.

In a technique of gas dissolution utilizing so-called bubbling, including hydrogen molecules dissolution, it has conventionally been considered that the important thing for producing a high-concentration gas solution is to make small the bubble size of the gas as much as possible, that is, to decrease the rising velocity of the bubbles toward the gas phase. Even at the time of filing of the present application, it still remains to be recognized as one of primary technical issues in the art to make micro-bubbles or nano-bubbles of various industrial gasses including hydrogen, oxygen and ozone.

In contrast, the present inventors have found that, in the opportunities in which consumers attempt to generate a high-concentration hydrogen-containing liquid when use it at various locations, such as home, workplace, street and storefront, it is far more desirable to first form the hydrogen gas phase in the tightly-closed container 30 and increase the inner pressure of the container 30 thereafter appropriately shaking the closed container 30 to recover the hydrogen gas from the gas phase, rather than directly dissolving hydrogen molecules into liquid including beverages such as drinking water, tea and coffee. Therefore, to increase the dissolved hydrogen concentration in the hydrogen-containing liquid, it is preferred, as illustrated in FIG. 3G, to put the capsule 20 of the present embodiment in the tightly-closed container 30 and appropriately shake the tightly-closed container 30.

The “shaking” in the present embodiment is to give a physical impact or shock to the tightly-closed container 30 thereby to replace the dissolved gas such as dissolved oxygen in the liquid L with hydrogen gas while bringing the liquid L in the tightly-closed container 30 into contact with the hydrogen gas in the gas phase. The shaking of the present embodiment involves natural shaking using hand or hands as well as artificial shaking using a machine. Examples of such artificial shaking include shaking by using a shaking machine, stirrer, ultrasonic generator, or other appropriate means. In order that the hydrogen gas is further accumulated in the gas phase of the tightly-closed container 30, the shaking may be started after the elapse of 1 minute, preferably 2 minutes, more preferably 4 minutes, further preferably 8 minutes, and particularly preferably 10 minutes, from the time when the capsule 20 is put into the tightly-closed container 30. To accelerate the dissolution of the high-pressure and high-concentration hydrogen gas into a living organism applicable liquid, the shaking time may be 5 seconds or longer, preferably 10 seconds or longer, more preferably 15 seconds or longer, and further preferably 30 seconds or longer, for the natural shaking. In consideration of easy shaking, the tightly-closed container may be provided therein with a head space of 15% or less, preferably 10% or less, and particularly preferably 5% or less, with respect to the container volume even after being filled with a living organism applicable liquid.

Through the method of using as the above, the hydrogen-containing liquid is obtained as illustrated in FIG. 3H.

FIG. 4 is a schematic view illustrating constitutional components of an apparatus 1 for generating a hydrogen-containing liquid according to another embodiment of the present invention and FIG. 5 is a view illustrating a method of using the apparatus 1 illustrated in FIG. 4 (corresponding to FIGS. 3D and 3E). The apparatus 1 illustrated in these figures represents an embodiment in which the capsule 20 is incorporated as a part of the container 30. Specifically, as illustrated in FIG. 4, the cap 32 of the container 30 is provided with the capsule 20 and, when the cap 32 is attached to the container 30 as illustrated in FIG. 5, the hydrogen gas is released from outside of the container 30 into the container 30. That is, the hydrogen gas is supplied into the container 30 even without putting the capsule 20 in the container 30 as illustrated in FIGS. 3D and 3E.

In the apparatus 1 of the above structure and method of using it, the present inventors were able to successfully generate a hydrogen-containing liquid having a dissolved hydrogen concentration of 7 ppm. Unfortunately, however, the present inventors were not able to generate a hydrogen-containing liquid having a hydrogen concentration higher than 8 ppm, in particular higher than 10 ppm, under the same condition. If the weight of the hydrogen generating agent 11 was increased, the generation amount of hydrogen gas would also increase and the dissolved hydrogen concentration could thus be increased, but an increased generation amount of hydrogen gas would increase the inner pressure in the container 30, as understood from the above-described method of use. In this case, therefore, a highly pressure-resistant container 30 would be required. Even when the weight of the hydrogen generating agent 11 was increased, an unreacted metallic material might have to be avoided from remaining, to reduce waste. As a result of intensive studies and trial and error in such a situation, the present inventors have successfully obtained a hydrogen-containing liquid of which the hydrogen concentration exceeds 8 ppm at an appropriate weight of the hydrogen generating agent 11 when the relationship between the weight W (number of moles can also be available) of the hydrogen generating agent 11 and the volume V of the capsule 20 (volume of the inside including the capsule main body 22, valve cap 23 and duckbill valve 21) is represented by a certain value. Examples of the present invention and comparative examples will be described below.

### EXAMPLES

### Example 1

Metal aluminum powder (available from Wako Pure Chemical Industries, Ltd., particle diameter of 53 to 150 μm, 80% up) and calcium hydroxide (available from Wako Pure Chemical Industries, Ltd.) as metallic materials were mixed at a ratio of 75 wt % of the metal aluminum powder and 25 wt % of the calcium hydroxide to obtain 0.66 g of a hydrogen generating agent 11. This 0.66 g of hydrogen generating agent was wrapped in a nonwoven fabric (Precise Regular C5160 available from Asahi Kasei Corporation), which was then heat-sealed to obtain a hydrogen generating body 10. A PET bottle for carbonated drink 30 having a volume of about 530 cc when filled with water fully to the mouth part was prepared. This PET bottle 30 was fully filled with Fujisawa city tap water (water temperature of 14.6° C.).

A capsule 20 having an inner volume of 5.4 ml was prepared. After the above hydrogen generating body 10 was immersed in the tap water in the PET bottle 30 for five to six seconds to be wet ted as illustrated in FIG. 3B, this hydrogen generating body 10 was put into a capsule main body 22, which was then closed with a valve cap 23. Thereafter, as illustrated in FIG. 3D, the capsule 20 was put into the PET bottle 30, which was then closed with a cap 32. Six sets of the same were prepared.

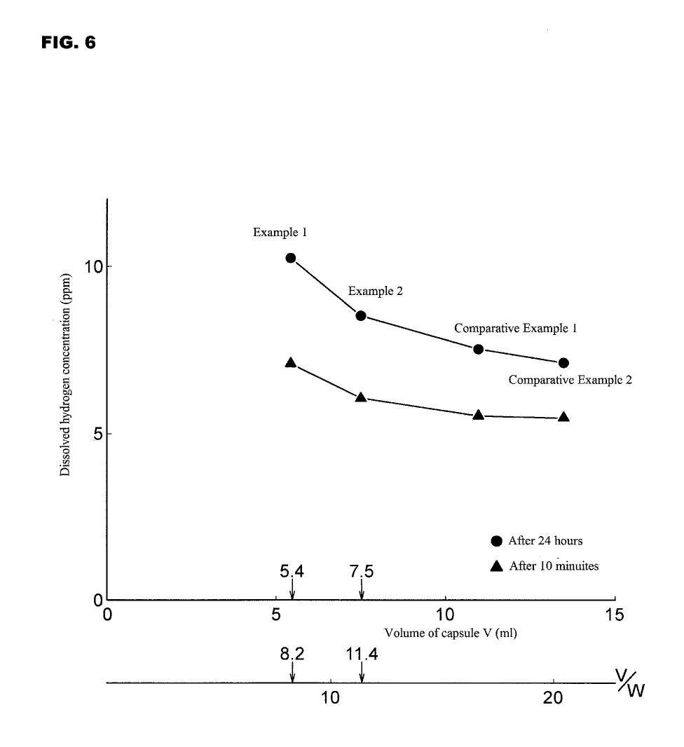

After the PET bottles were left for 10 minutes and 24 hours, one of the present inventors (Japanese man of 30s having an average physical size) held the middle portion of each PET bottle by his dominant hand and moved only the wrist to right and left to shake it so that the cap was forming into an arch above the wrist with a pace of 2 strokes per second, total 120 strokes (total 60 seconds). Thereafter, the dissolved hydrogen concentration in each content liquid L was measured. Results are listed in Table 1 and illustrated in FIG. 6. The dissolved hydrogen concentration was titrated with a dissolved hydrogen concentration measuring reagent (9.88 ml of alcohols containing ethanol, methylene blue and colloidal platinum) available from MiZ Company Limited.

### Example 2

The dissolved hydrogen concentration was measured for the content liquid L obtained under the same condition as in the above Example 1 except that the inner volume of the capsule 20 was 7.5 ml. Results are listed in Table 1 and illustrated in FIG. 6.

### Comparative Example 1

The dissolved hydrogen concentration was measured for the content liquid L obtained under the same condition as in the above Example 1 except that the inner volume of the capsule 20 was 11.0 ml. Results are listed in Table 1 and illustrated in FIG. 6.

### Comparative Example 2

The dissolved hydrogen concentration was measured for the content liquid L obtained under the same condition as in the above Example 1 except that the inner volume of the capsule 20 was 13.5 ml. Results are listed in Table 1 and illustrated in FIG. 6.

TABLE 1

| | | | Dissolved | Dissolved |
|---|---|---|---|---|
| | | | hydrogen | hydrogen |
| | Capsule | | concentration | concentration |
| | volume | | after 10 | after 24 |
| | V (ml) | V/W | minutes (ppm) | hours (ppm) |
| Example 1 | 5.4 | 8.2 | 7.1 | 10.2 |
| Example 2 | 7.5 | 11.4 | 6.0 | 8.5 |
| Comparative | 11.0 | 16.7 | 5.5 | 7.5 |
| Example 1 | | | | |
| Comparative | 13.5 | 20.5 | 5.4 | 7.1 |
| Example 2 | | | | |

W = 0.66 g

### <<Consideration<<

Taking the rate of change (first-order derivative) of the dissolved hydrogen concentration in the above Examples 1 and 2 and Comparative Examples 1 and 2, a significant difference is found between Example 2 and Comparative Example 1 in the dissolved hydrogen concentration after 10 minutes and a significant difference is found between Example 1 and Example 2 in the dissolved hydrogen concentration after 24 hours. Therefore, in order that the dissolved hydrogen concentration after 10 minutes is 6 ppm or higher, it is preferred that the ratio (V/W) of a volume (V ml) of the capsule 20 to a weight (W g) of the hydrogen generating agent 11 be 11.4 or less. In this case, the dissolved hydrogen concentration after 24 hours is to exceed 8 ppm. In order that the dissolved hydrogen concentration after 24 hours is 10 ppm or higher, it is preferred that the ratio (V/W) of a volume (V ml) of the capsule 20 to a weight (W g) of the hydrogen generating agent 11 be 8.2 or less.

### Example 3

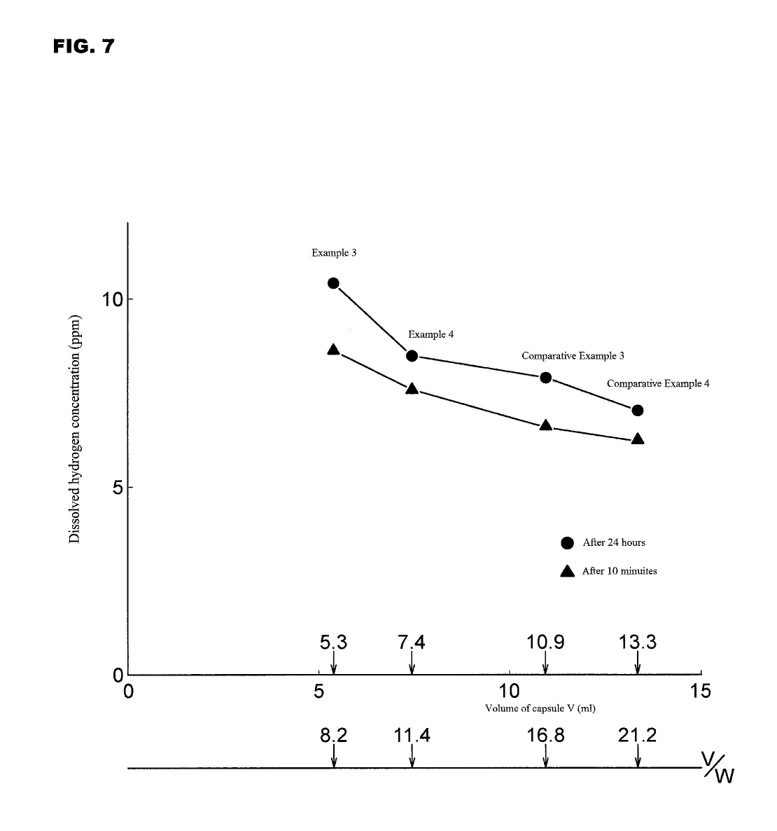

A hydrogen-containing liquid was generated under the same condition as in Example 1 except that the amount of hydrogen generating agent 11 of the hydrogen generating body 10 was 0.65 g, the volume of PET bottle for carbonated drink 30 when filled with water fully to the mouth part was 300 ml, and the inner volume of the capsule 20 was 5.3 ml. The dissolved hydrogen concentration of the content liquid L thus obtained was measured. Results are listed in Table 2 and illustrated in FIG. 7.

### Example 4

The dissolved hydrogen concentration was measured for the content liquid L obtained under the same condition as in the above Example 3 except that the inner volume of the capsule 20 was 7.4 ml. Results are listed in Table 2 and illustrated in FIG. 7.

### Comparative Example 3

The dissolved hydrogen concentration was measured for the content liquid L obtained under the same condition as in the above Example 3 except that the inner volume of the capsule 20 was 10.9 ml. Results are listed in Table 2 and illustrated in FIG. 7.

### Comparative Example 4

The dissolved hydrogen concentration was measured for the content liquid L obtained under the same condition as in the above Example 3 except that the inner volume of the capsule 20 was 13.3 ml. Results are listed in Table 2 and illustrated in FIG. 7.

TABLE 2

| | | | Dissolved | Dissolved |
|---|---|---|---|---|
| | | | hydrogen | hydrogen |
| | Capsule | | concentration | concentration |
| | volume | | after 10 | after 24 |
| | V (ml) | V/W | minutes (ppm) | hours (ppm) |
| Example 3 | 5.3 | 8.2 | 8.6 | 10.3 |
| Example 4 | 7.4 | 11.4 | 7.6 | 8.4 |
| Comparative | 10.9 | 16.8 | 6.6 | 7.8 |
| Example 3 | | | | |
| Comparative | 13.3 | 21.2 | 6.2 | 7.0 |
| Example 4 | | | | |

W = 0.65 g

### <<Consideration<<

Taking the rate of change (first-order derivative) of the dissolved hydrogen concentration in the above Examples 3 and 4 and Comparative Examples 3 and 4, a significant difference is found between Example 4 and Comparative Example 3 in the dissolved hydrogen concentration after 10 minutes and a significant difference is found between Example 4 and Comparative Example 3 in the dissolved hydrogen concentration after 24 hours. Therefore, in order that the dissolved hydrogen concentration after 10 minutes is 6 ppm or higher, it is preferred that the ratio (V/W) of a volume (V ml) of the capsule 20 to a weight (W g) of the hydrogen generating agent 11 be at least 11.4 or less. In this case, the dissolved hydrogen concentration after 24 hours is to exceed 8 ppm. In order that the dissolved hydrogen concentration after 24 hours is 10 ppm or higher, it is preferred that the ratio (V/W) of a volume (V ml) of the capsule 20 to a weight (W g) of the hydrogen generating agent 11 be 8.2 or less.

### DESCRIPTION OF REFERENCE NUMERALS

1 Apparatus for generating hydrogen-containing liquid

10 Hydrogen generating body

11 Hydrogen generating agent

12 Bag body

20 Capsule

21 Duckbill valve (one-way valve, return check valve, or gas-permeable film)

22 Capsule main body

23 Valve cap

24 Projection

25 Slit

30 Container

31 Container main body

32 Cap
L Liquid in which hydrogen gas is to be contained

What is claimed is:

1. An apparatus for generating a hydrogen-containing liquid, the apparatus comprising:
a hydrogen generating agent reacting with water to generate hydrogen gas;
a capsule configured to be charged with the hydrogen generating agent and discharge the hydrogen gas generated in the capsule to external; and
a container configured to be charged with a liquid that is an object to which the hydrogen gas generated in the capsule is added,
wherein a ratio (V/W) of a volume (V ml) of the capsule to a weight (W g) of the hydrogen generating agent is 11.4 or less.

2. The apparatus according to claim 1, wherein the ratio is 8.2 or less.

3. The apparatus according to claim 1, wherein
the hydrogen generating agent contains a metallic material having a higher ionization tendency than hydrogen or a hydrogenated compound and an accelerant, and
the capsule is charged with the hydrogen generating agent in a state of being put in a bag body that is permeable to water.

4. The apparatus according to claim 1, wherein
the capsule has a bottom-closed tubular capsule main body that is to be charged with the hydrogen generating agent and a valve cap that closes an opening of the capsule main body, and
a projection is provided at an outer surface of the capsule main body or valve cap so as to project outward from the outer surface.

5. The apparatus according to claim 1, wherein the container has a volume of 300 ml to 530 ml.

6. A method for generating a hydrogen-containing liquid using the apparatus according to claim 1, the method comprising:
charging the container with a liquid that is to contain hydrogen;
soaking the hydrogen generating agent in water;
charging inside of the capsule with the hydrogen generating agent;
putting the capsule into the container and tightly closing the container; and
leaving the container for a predetermined time.

7. The method according to claim 6, wherein the container is shaken after the container is left for the predetermined time in a state of being tightly closed.

8. A kit for generating a hydrogen-containing liquid, the kit comprising:
a hydrogen generating agent reacting with water to generate hydrogen gas; and
a capsule configured to be charged with the hydrogen generating agent and discharge the hydrogen gas generated in the capsule to external,
wherein the kit is used for charging a container with a liquid that is an object to which the hydrogen gas generated in the capsule is added and generating the hydrogen-containing liquid, and
a ratio (V/W) of a volume (V ml) of the capsule to a weight (W g) of the hydrogen generating agent is 11.4 or less.
